(19) United States
(12) Patent Application Publication
(10) Pub. No.: US 2011/0104013 A1
(43) Pub. Date: May 5, 2011

(54) METHOD FOR PRODUCING PHOTOCATALYTIC MATERIALS AND MATERIALS AND APPARATUS THEREWITH

(75) Inventor: Jose Barreto, Fort Myers, FL (US)

(73) Assignee: FLORIDA GULF COAST UNIVERSITY, Fort Myers, FL (US)

(21) Appl. No.: 12/994,215

(22) PCT Filed: May 18, 2009
(86) PCT No.: PCT/US2009/044304

§ 371 Date: Nov. 23, 2010

Related U.S. Application Data
(60) Provisional application No. 61/056,257, filed on May 27, 2008.

(51) Int. Cl. B01J 19/12; B01J 21/08; B01J 37/08; B01J 37/02

(52) U.S. Cl. 422/122; 502/242; 422/186.3

(57) ABSTRACT

Methods for the production of photocatalytic materials and coated substrate materials are provided according to the invention. A process for forming a photocatalytic coating on a substrate is provided. The photocatalytic coating includes titanium oxide on a titanium substrate for example. The photocatalytic coated substrate may be used in a device for treating contaminated fluids including water, air and mixtures thereof.

### CROSS REFERENCE TO RELATED APPLICATION

This patent application is the national stage application of international application PCT/US09/44304, filed May 18, 2009, which claims priority to and the benefit of U.S. Provisional Application Ser. No. 61/056,257 filed on May 27, 2008, the entire disclosure of which is hereby incorporated by reference.

### GRANT REFERENCE

The research carried out in connection with this invention was supported in part by a grant from the Department of Defense [DOD-N00173-07] and the Environmental Protection Agency [EM-83298201-1]. The Government has certain rights in the invention.

### FIELD OF THE INVENTION

The invention relates to methods for producing photocatalytic materials and coatings, and uses and apparatus therefore. More particularly, the invention relates to methods for producing a photocatalytic coating on a substrate, or photocatalytic materials for use in various applications, wherein the photocatalytic materials are a titanium oxide material.

### BACKGROUND OF THE INVENTION

Photocatalysts are useful for a variety of applications, and a variety of photocatalytic materials have been developed. Photocatalysis is the acceleration of a photoreaction in the presence of a catalyst, and photocatalytic activity depends on the ability of the catalyst to create electron-hole pairs, which generate free radicals that may be used for various applications, and undergo secondary reactions. Although some materials provide photocatalytic activity, the activity may not be sufficient for useful application, and/or the creation of the material is complex and costly, making it difficult to use for many applications. For example, contaminants in fluid streams and the air, such as organic compounds, nitrogen and sulfur oxides, acid gasses, dissolved inorganic solids, and microorganisms are converted by the oxidizing and reducing potential of the photocatalytic material formed of an activated semiconductor. The conversion may take the form of, but is not limited to, the oxidation of organic compounds, degradation of microorganisms, or reduction of dissolved ionic species. The products of the conversion are ideally less harmful or more easily removed from the fluid stream than the parent compounds.

Semiconductor photocatalysts that have been demonstrated for the destruction of organic contaminants in fluid media include but are not limited to: TiO₂, ZrO₂, ZnO, CaTiO₃, SnO₂, MoO₃, Fe₂O₃, and WO₃.

Titanium dioxide has been employed as a photocatalyst in air purifiers to destroy contaminants. When the titanium dioxide is illuminated with ultraviolet light, photons are absorbed by the titanium dioxide, promoting an electron from the valence band to the conduction band, thus producing a hole in the valence band and adding an electron in the conduction band. The promoted electron reacts with oxygen, and the hole remaining in the valence band reacts with water, forming reactive hydroxyl radicals. When a contaminant adsorbs onto the titanium dioxide photocatalyst, the hydroxyl radicals attack and oxidize the contaminants to water, carbon dioxide, and other substances.

Metal oxide/titanium dioxide photocatalysts have been employed as a water-phase photocatalyst to remove contaminants from a water flow. Water phase chemistry is significantly different from air phase chemistry. The reaction mechanisms are in general different in each phase. Therefore, a catalyst designed for aqueous phase chemistry does not perform in the same manner as a catalyst designed for gas phase chemistry. Additionally, the hydroxyl radical can diffuse away from the photocatalyst surface in an aqueous phase, and the hydroxy radical does not diffuse away from the photocatalyst surface in the gas phase. Finally in the water phase, the water and ionic species in the water compete with the contaminants for adsorption sites on the photocatalyst, also reducing the photocatalytic performance.

Sol-gel coating processes have been employed to create a photocatalytic suspension that is applied to a substrate to create a photocatalytic coating. The sol-gel coating process achieves the desired photocatalytic loading and high adhesion performance through multiple dip coating processes. A drawback to the sol-gel coating process is that it requires expensive titanium precursors (such as titanium isopropoxide) and complicated reflux/sonication procedures. Therefore, the sol-gel coating process is costly and labor intensive.

It would be advantageous to provide methods of forming photocatalytic materials and photocatalytic coated metal substrates, such as for use for the decontamination of fluids that can be accomplished in a straightforward and cost-effective manner.

### SUMMARY OF THE INVENTION

In general, one example of the invention is to provide methods or processes for producing photocatalytic materials. A process for preparing a photocatalytic material according to the invention may comprise the steps of providing a metal substrate and subjecting the metal substrate to an acid mixture. Heating of the metal substrate and acid mixture to a predetermined temperature for a predetermined amount of time forms an acid treated metal substrate. Neutralizing the acid-treated metal substrate with a basic solution, and annealing the neutralized metal substrate at a predetermined temperature for a predetermined amount of time forms a coating on the metal substrate formed of a photocatalytic material.

In yet another aspect of the invention, a photocatalytic coated substrate includes a metal substrate having a first surface and a second surface, and a photocatalytic coating covering at least a portion of the first surface of the metal substrate, wherein the photocatalytic coating comprises a metal oxide coating, wherein the metal oxide coating is formed from metal ions in the metal substrate.

In another embodiment of the invention, a device for decontaminating a fluid includes at least one container comprising a sealed interior portion, an inlet providing access to the sealed interior portion and an outlet providing an exit from the sealed interior portion, wherein the sealed interior portion includes a ultra-violet source of light and a metal substrate coated with a photocatalytic coating.

Another embodiment of the invention relates to photocatalytic coatings including semi-conducting materials based on metal oxide, in particular on titanium oxide, which are capable of initiating radical reactions under the effect of radiation of appropriate wavelength, resulting in the oxidation of organic products. These coatings thus make it possible to confer novel functionalities on the materials, which they cover, in particular fungicidal, bactericidal, algicidal or odor-controlling properties.

### BRIEF DESCRIPTION OF THE DRAWINGS

FIG. 1 is a process diagram illustrating an embodiment of a method of preparing a photocatalytic coating on a substrate;

FIG. 2 is a graph representing THA fluorescence measurement showing photocatalytic effectiveness for a variety of samples formed according to the process of the invention;

FIG. 3 is a graph representing Tartrazine absorbance for mean standard deviations for a plurality of samples formed according to the process of the invention;

FIG. 4 is a graph representing Sudan absorbance for mean standard deviations for a plurality of samples formed according to the process of the invention;

FIG. 5 is a graph representing measurement of hydroxyl radical production of photocatalytic coatings according to one embodiment of the invention;

FIG. 6 is a graph representing measurement of hydroxyl radical production of photocatalytic coatings after various annealing temperatures according to one embodiment of the invention;

FIG. 7 is a cross-sectional schematic illustration of a first device for decontaminating fluids according to one embodiment of the invention; and

FIG. 8 is a cross-sectional schematic illustration of a second device for decontaminating fluids according to one embodiment of the invention.

### DETAILED DESCRIPTION OF THE INVENTION

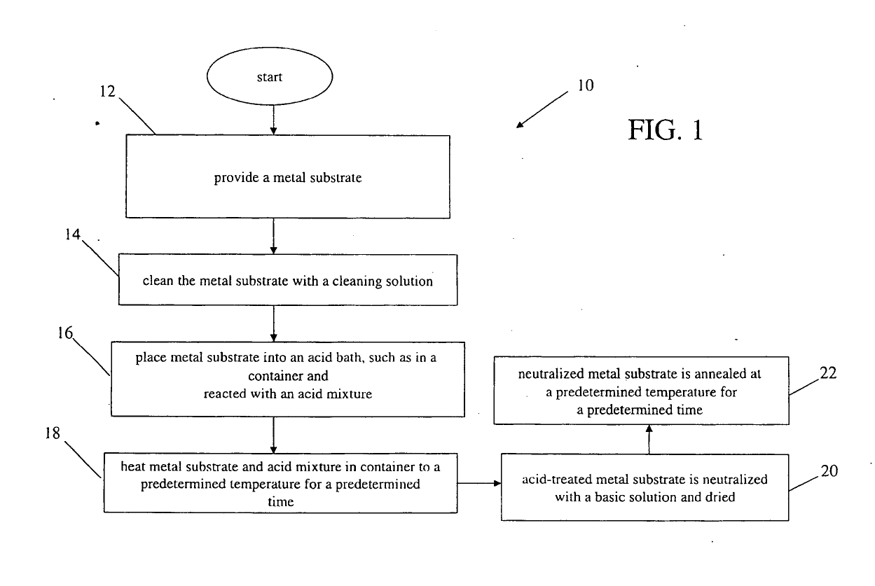

Turning to a first example of a method according to the invention, FIG. 1 illustrates a process diagram of a method of forming a photocatalytic coating generally designated 10, and furthermore, the method for preparing a photocatalytic coating on a substrate. In a first step at 12, a metal substrate is provided. In one embodiment of the invention, the metal substrate may include a metal foil, metal wool and a powdered metal material. In an example, the metal substrate is titanium metal. In yet another embodiment of the invention, the metal substrate is a titanium alloy. Next, in step 14, the metal substrate may be cleaned with a cleaning solution, although such a process may not be necessary depending on the characteristics of the substrate. In one example, the cleaning solution may include water, a polyol, an acid and a metal fluoride. In yet another embodiment of the invention, the cleaning solution may include water, ethylene glycol, hydrochloric acid and a sodium fluoride. Any other suitable cleaning solution may be used. Next, as described in step 16, the metal substrate is placed into an acid bath in a suitable container and reacted with an acid mixture. In an embodiment of the invention, the container is made of glass. In yet another embodiment of the invention, the glass container is made of borosilicate glass. In general, the container is formed with a material having a silicate component, such as silicon oxide found in glass materials. In an example of the invention, the acid mixture is a combination of hydrochloric acid and nitric acid. More particularly, the acid may be a mixture of hydrochloric and nitric acids in a ration of between 25-75%:75-25% vol/vol respectively, or between 40-60%:60-40% vol/vol for example, or according to a further example 45% HCl: 55% HNO₃ vol/vol. The mixture of hydrochloric acid and nitric acid has been found to produce the desired photocatalytic activity, but other suitable acids or acid mixtures may also be used. In a further example of the invention, the acid mixture may include a sodium fluoride.

In step 18, the metal substrate and acid mixture is heated, such as heating to at least a predetermined temperature for a predetermined amount of time. In an example, the substrate and acid mixture is heated to at least about between 100° C. to 400° C. for at least about between 15 minutes to one hour. In a more particular example, the acid mixture is heated to about 150° C. to 250° C. for about 20 to 40 minutes, or in another example the substrate and acid mixture is placed in a sand bath at about 200° C. for about 30 minutes. Other temperatures and/or times may be suitable. Next, at step 20, the acid-treated metal substrate is neutralized with a basic solution and dried. Finally, as described in step 22, the neutralized metal substrate is annealed at a predetermined temperature for a predetermined amount of time. The predetermined temperature in an example may be between 200° C. to 800° C., and the predetermined time may be 15 minutes to two hours for example. In more particular examples, the annealing is carried out at a temperature of between about 500° C. to about 700° C. for about between 30 minutes to one and one half hours, or at a temperature of about 600° C. for about one hour. Other temperatures and/or times may be suitable. In another example, the substrate may be exposed to an annealing temperature where the temperature is increased at a ramp rate of 10° C. per minute to the predetermined temperature.

### Example 1

In a particular example according to the invention, a 0.025 mm thick titanium foil measuring 2 cm×3 cm was placed into a cleaning solution composed of 2.5 ml H₂O, 3.0 ml ethylene glycol, 4.5 ml 2M HCl and 100 mg NaF until the surface was dull and covered with fine bubbles (approximately 3 minutes) at room temperature. The foil was removed from cleaning solution and placed directly into 9 ml of concentrated HCl in a 100 ml Pyrex beaker. 11 ml of concentrated HNO₃ (Acros, 25 ml/bottle) was added along with 20 mg. of NaF. The beaker was swirled to mix the contents. The beaker was then placed into a 200° C. sand bath and covered with a watch glass. Any suitable heating arrangement may be used. The reaction was allowed to proceed for 30 minutes. The titanium is bright silver in color when first placed into the acid bath described above; after 30 minutes in the acid bath, it was noted that a dark grey coating had formed on both sides of the foil and a milky solution was present. The foil was placed into a saturated solution of Na₂CO₃ to neutralize the acid followed by a tap water rinse and rub to remove the milky solution from the foil. The foil was rinsed with DI water and allowed to air dry. The air dried foil is calcined at 600° C. for 1 hour in a muffle furnace. To support the effect of having an amount of silicon oxide in the composition of the coating to enhance the photocatalytic effectiveness, in an alternative to the above described process, a Teflon beaker was substituted for the Pyrex beaker described above, which resulted in a marked reduction in the photocatalytic activity of the resulting foil. This indicates that silicon oxide from the glass appears to be a component of the photocatalytic coating. An amount of silicon oxide may be provided from any suitable source, and may be in the range of 0.001 to 5% by weight in the mixture for example. Various other combinations of HCl and HNO₃ yield active coatings but the ratio of 45% HCl:55% HNO₃, vol/vol appeared to yield higher generation of an active photocatalyst.

The formation of titanium (IV) oxide, along with an amount of silicon oxide, on the surface of the treated metal substrate provides the photocatalytic coating that may be used in association with the substrate, or removed from the substrate to form a photocatalytic powder type of material for use in other applications. Titanium oxide photocatalysts according to the invention have many possible uses. When the titanium oxide coating formed according to the invention is illuminated with ultraviolet light (or potentially visible light), electrons and positive holes are generated and these electrons and positive holes can be utilized for many purposes. Some examples of general uses for the formed photocatalysts include where the reductive electrons and oxidative positive holes can be used to synthesize molecules. For example, the titanium oxide photocatalysts may be used in the production of methanol from carbon dioxide to create a liquid transportation fuel, using sunlight. In another example, the photocatalytic coatings and/or powders could also be used to create sensors to detect certain analytes. If there is an interaction between the analyte and the surface, changes in conductance or resistance can be used to detect the presence of the analyte. Further, the oxidative, reductive and radical species generated by photocatalysts according to the invention can be used to decontaminate air and water.

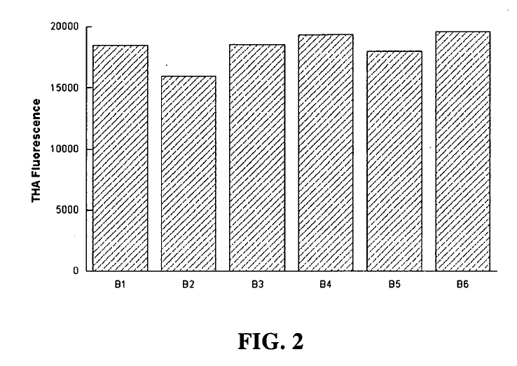

Turning to FIG. 2, the photocatalytic effectiveness of the materials formed according to examples of the invention are evaluated. Terephthalic acid (THA) is a water soluble, non-fluorescent compound, but when hydroxylated by hydroxyl radicals, the THA-OH becomes intensely fluorescent, generating an easily measurable signal, (Barreto et al, Life Sciences Vol. 56 (4), p. 89-96 1995). To evaluate the materials produce, a THA “dosimeter” is used to screen for photocatalytic effectiveness since hydroxyl radicals are one type of destructive decontaminating species produced by photocatalysis. In an experiment, large values for fluorescence indicate high photocatalytic activity. As seen in FIG. 2, B1-B6 represent six individual photocatalytic foils having a coating formed thereon according to the process of Example 1 above. All six foils exhibit high signal generation as reported by the THA dosimeter, meaning that many hydroxyl radicals were present in the solution and that the photocatalysts were very active. An untreated foil showed a signal of 150 to 300 units (data not shown). As indicated, the coating generated according to the invention produces a signal on the order of 15,000 to 17,000 units THA-OH fluorescence units.

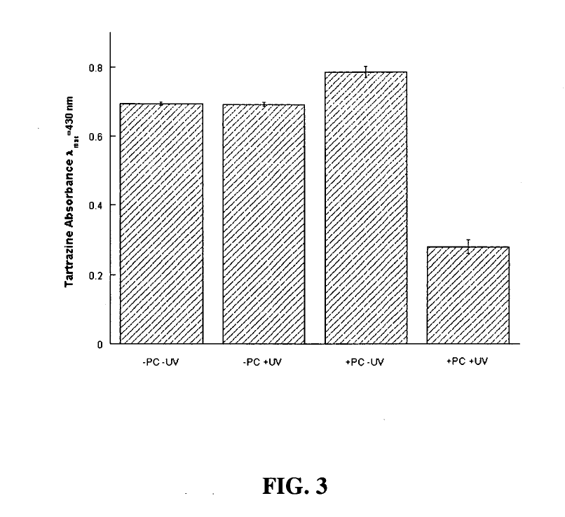

In another example of evaluating the material as produced according to the invention, tartrazine is a water soluble azo dye which can be photobleached by radicals produced during photocatalysis. As seen in FIG. 3, evaluation using such an approach is shown. In the data of dye photobleaching shown in FIG. 3, the results serve a as a model for water soluble toxin destruction. FIG. 3 shows the photocatalytic destruction of tartrazine yellow by chemically treated foils produced according to the Example 1. A decrease in absorbance indicates dye destruction, so that the lowest bar in the figure indicates the most destruction. +UV indicates ˜5 mW/cm² long wave UV light. +PC indicates the presence of individual B1-B6 photocatalytic foils (the same B1-B6 foils described in FIG. 2). The Mean±standard deviations are shown for six separate trials (B1-B6), in the graph of FIG. 3. −PC is a control indicating the absence of a foil; −UV indicates no light. Thus, −PC −UV in FIG. 3 shows that no treatment at all has no effect on dye destruction. −PC +UV shows that the dye is very resistant to UV photolysis alone, which this is an indicative control because photobleaching of the dye without photocatalyst would cause complications in the interpretation of the photobleaching caused by photocatalysis. Further, it is shown that the materials formed according to the invention provide destruction of a very UV resistant target. +PC −UV shows an anomalously high absorbance which may be due to a change in pH upon addition of the foil (Tartrazine absorbance is pH sensitive). The only experiment to show a significant destruction is +PC +UV indicating that both light and photocatalyst provide dye destruction and that the process for producing chemically treated foils according to the invention produces very effective photocatalysts.

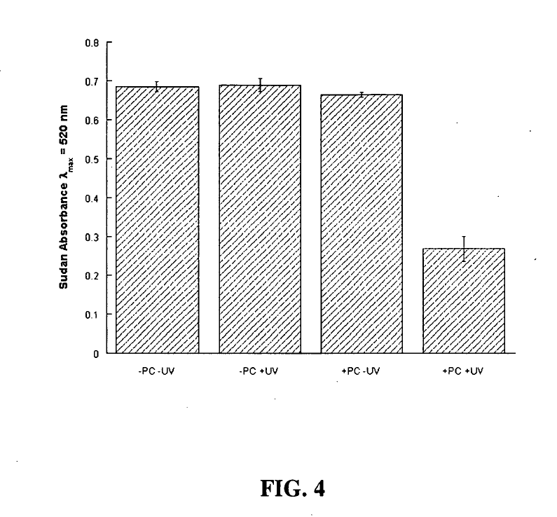

Turning to FIG. 4, there is shown a measure of absorbance of Sudan red by the materials formed according to the invention. In FIG. 4, the indications of +−PC +−UV have the same meanings as described in FIG. 3. Sudan red is a water insoluble azo dye which has been encapsulated inside a CTAB detergent micelle. Sudan red photobleaching is an assay for the destruction of a toxin target encapsulated in a membrane (a hydrophobic dye). Although the Sudan red was encapsulated, it could be photobleached by the photocatalytic radicals, oxidants or reductants produced by the coated foils formed according to the Example 1. FIG. 4 shows the photocatalytic destruction of Sudan red by the chemically treated foils, as measured by a decrease in absorbance, so that the lowest bar in the figure indicates the most destruction. −PC −UV indicates that no treatment has no effect on Sudan dye destruction. −PC +UV shows that the Sudan is very resistant to UV photolysis alone, again, being an indicative control because photobleaching of the dye without photocatalyst would cause complications in the interpretation of photobleaching caused by photocatalysis. Unlike tartrazine, Sudan red does not experience any absorbance change upon the addition of the foil to the dye solution +PC −UV, does not affect Sudan red absorbance. The only experiment to show a significant destruction is +PC +UV indicating that both light and photocatalyst achieve destruction, and that the process for producing chemically treated foils according to the invention is effective as a photocatalyst. The Mean±standard deviation shown of six separate trials with B1-B6 foils in each group are shown in FIG. 4.

Accordingly, it is apparent based on the evaluation of the materials produced according to the invention that when illuminated with ultra-violet (UV) light, the titanium oxide is capable of producing free radicals, oxidants and reductants that may function as powerful biocides and/or a toxin destroyer as an example. In one embodiment of the invention, the titanium oxide photocatalytic coating may be illuminated with an 8 watt medium pressure mercury lamp with a primary emission of 365 nm. The fluorescence of the photocatalytic coating is read with an Aminco Bowman fluorometer at an excitation of 312 nm and an emission of 426 nm.

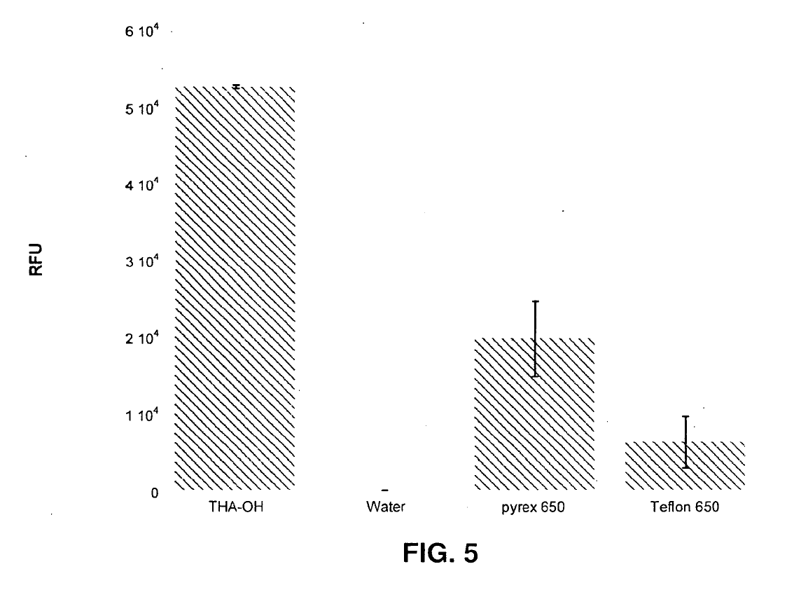

As seen in FIG. 5, the measurement of relative fluorescence units (RFU) of the production of hydroxyl radical production was performed on an acid treated titanium foil in a Pyrex® glass container and compared with an acid treated titanium foil in a Teflon® coated container. A hydroxylated terephthalic acid material was used as a standard in this dosimetry measurement. As mentioned above, terephthalic acid (THA) is non-fluorescent, but when hydroxylated by (.OH) at any of its four identical aromatic ring positions, the compound (THA-OH) becomes highly fluorescent. As seen in FIG. 5, there is a clear difference in the measurement of hydroxyl radicals formed when the metal substrate is reacted with the acid mixture in the Pyrex® glass container when compared when the metal substrate is reacted with the acid mixture in the Teflon® coated container. FIG. 5 shows that the use of the Pyrex® glass container provides almost five times the hydroxyl radical production when compared to the use of the Teflon® coated container.

Energy Dispersive X-Ray Spectroscopy (EDAX) analysis on the photocatalytic coating shows that the coating is comprised of titanium oxide with a trace of silicon oxide. It is known that glass is composed of silicon oxide. Glass is a relatively inert material, which is why many chemists use these types of containers for a multitude of reactions, but it was determined that conditions were created wherein the glass container was being partially dissolved and then interacting with the photocatalytic coating. The discovery of this “glass effect” was determined to be accomplished through the addition of the sodium fluoride in the acid mixture under highly acidic conditions. The combination of the sodium fluoride and the acid mixture produce hydrofluoric acid which etches the wall of the glass container. It is believed that the dissolved glass, i.e. silicon oxide, becomes a component of the photocatalytic coating which facilitates the production of the hydroxyl radicals. Furthermore, the use of the Teflon® coated container, which is inert to hydrofluoric acid and is devoid of silicon, is found to produce an inferior photocatalytic coating as seen by the lower production of hydroxyl free radicals. Thus, an amount of silicon oxide as a component of the photocatalytic coating provides enhanced photocatalytic activity.

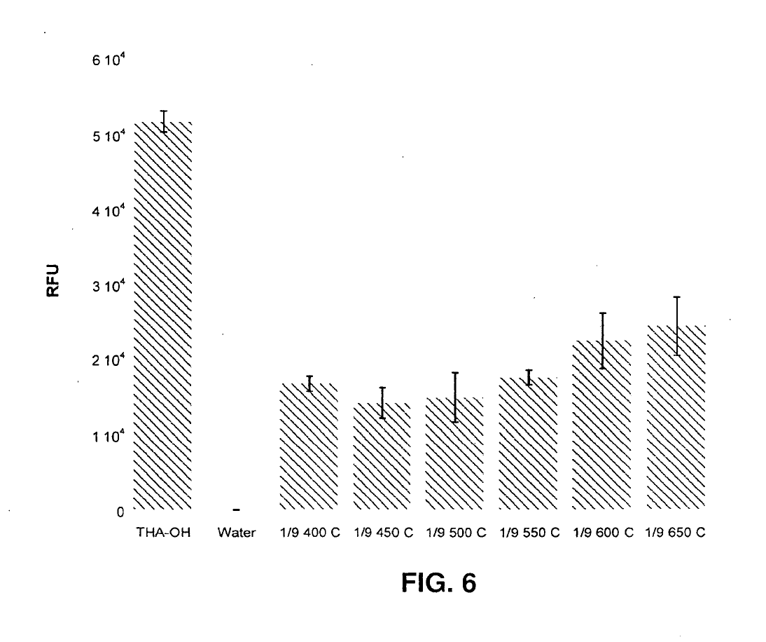

As seen in FIG. 6, the measurement of relative fluorescence units (RFU) of the production of hydroxyl radical production was performed on an acid treated titanium foil and then subjected to various annealing temperatures. It was determined that an annealing temperature of 650° C. was the optimal temperature for the production of hydroxyl free radicals when compared to the THA-OH.

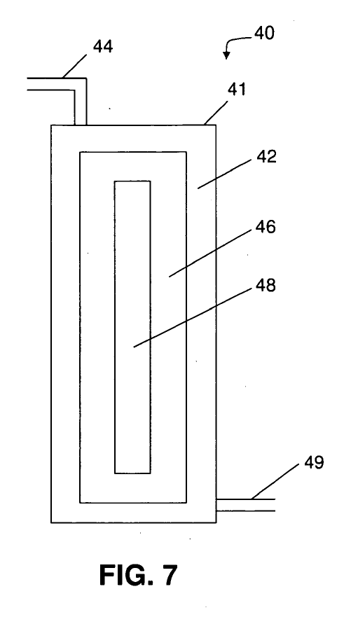

In another embodiment of the invention, as seen in FIG. 7, a device 40 for decontaminating a fluid is disclosed. Decontamination of fluids, including water and air, presents a myriad of challenges. The biocide or decontaminating agent should destroy all of the chemical and biological hazards found in the environment that it contacts. However, persistent biocides, such as chlorine for example, have their limitations since they pose a long term hazard to cultured organisms. A viable decontamination agent should be lethal enough so that substantially complete decontamination may take place when contacting contaminated fluids. Sanitization with photocatalysis may be a viable way to decontaminate fluids since the sanitizing species created, which include superoxide, hydroxyl and organic free radicals, each have very short lifetimes and decay rapidly into relatively harmless substances. Device 40 includes a container 41 that includes a sealed interior portion 42. Inlet 44 provides access of a fluid, which includes contaminated water, air or mixtures thereof, into sealed interior portion 42 of device 40. Within sealed interior portion 42 is a photocatalytic coated metal substrate 46 and a UV light source 48. In one embodiment of the invention, photocatalytic coated metal substrate 46 may include a metal foil, metal wool and a powdered metal. The contaminated water, air or mixtures thereof are subjected to reductive and oxidative chemical species that are formed when the UV light produced by UV light source 48 is exposed to photocatalytic coated metal substrate 46. In an embodiment of the invention, sealed interior portion 42 may also include at least one mirror or reflective surface to reflect unabsorbed photons that are produced when the UV light is exposed to photocatalytic coated metal substrate 46. The reductive and oxidative chemical species destroy substantially all of the toxins and pathogens found in the contaminated water, air or mixtures thereof introduced into sealed interior portion 42 of device 40. After exposure to the reductive and oxidative chemical species, the decontaminated water, air or mixtures thereof flow out of device 40 through outlet 49. Although not shown, a power source provides energy to UV light source 48. In one embodiment, the power source is an electrical outlet. In yet another embodiment the power source is a battery which may be rechargeable.

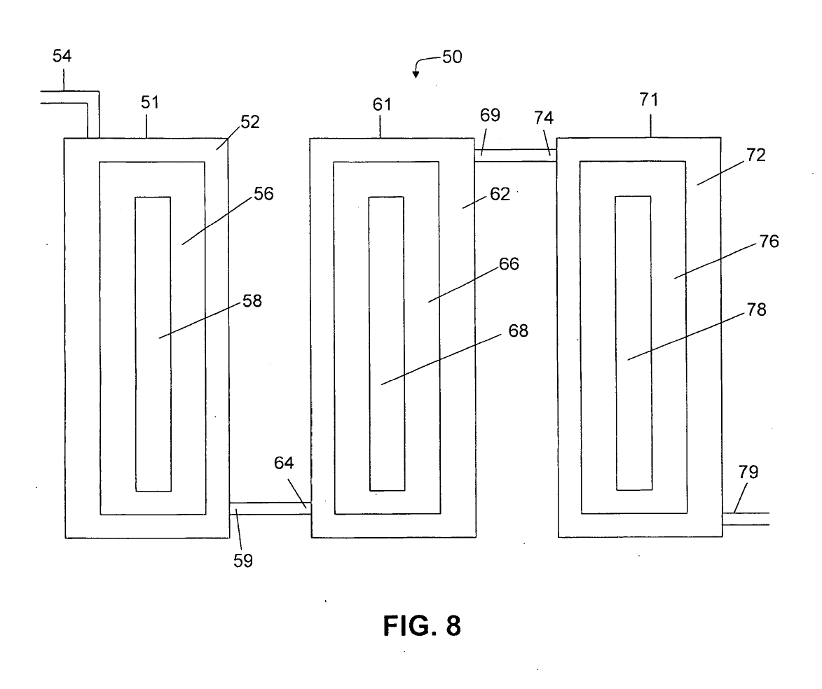

In yet another embodiment of the invention, as seen in FIG. 8, a device 50 for decontaminating a fluid is disclosed. Device 50 includes a first container 51 that includes a sealed interior portion 52. Inlet 54 provides access of a fluid, which includes contaminated water, air or mixtures thereof, into sealed interior portion 52 of container 51. Within sealed interior portion 52 is a photocatalytic coated metal substrate 56 and a UV light source 58. In one embodiment of the invention, photocatalytic coated metal substrate 56 may include a metal foil, metal wool and a powdered metal. The contaminated water, air or mixtures thereof are subjected to reductive and oxidative chemical species that are formed when the UV light produced by UV light source 58 is exposed to photocatalytic coated metal substrate 56. In an embodiment of the invention, scaled interior portion 52 may also include at least one mirror to reflect unabsorbed photons that are produced when the UV light is exposed to photocatalytic coated metal substrate 56. The reductive and oxidative chemical species destroy substantially all of the toxins and pathogens found in the contaminated water, air or mixtures thereof introduced into sealed interior portion 52 of container 51. After exposure to the reductive and oxidative chemical species, the decontaminated water, air or mixtures thereof flow out of container 51 through outlet 59 and into second container 61 through inlet 64. Second container 61 includes a sealed interior portion 62 and inlet 64 provides access of a fluid, which includes contaminated water, air or mixtures thereof, into sealed interior portion 62 of container 61. Within sealed interior portion 62 is a photocatalytic coated metal substrate 66 and a UV light source 68. In one embodiment of the invention, photocatalytic coated metal substrate 66 may include a metal foil, metal wool and a powdered metal. The contaminated water, air or mixtures thereof are subjected to reductive and oxidative chemical species that are formed when the UV light produced by UV light source 68 is exposed to photocatalytic coated metal substrate 66. In an embodiment of the invention, sealed interior portion 62 may also include at least one mirror to reflect unabsorbed photons that are produced when the UV light is exposed to photocatalytic coated metal substrate 66. The reductive and oxidative chemical species destroy substantially all of the toxins and pathogens found in the contaminated water, air or mixtures thereof introduced into sealed interior portion 62 of container 61. After exposure to the reductive and oxidative chemical species, the decontaminated water, air or mixtures thereof flow out of container 61 through outlet 69 and into third container 71 through inlet 74. Third container 71 includes a sealed interior portion 72 and inlet 74 provides access of a fluid, which includes contaminated water, air or mixtures thereof, into sealed interior portion 72 of container 71. Within sealed interior portion 72 is a photocatalytic coated metal substrate 76 and a UV light source 78. In one embodiment of the invention, photocatalytic coated metal substrate 76 may include a metal foil, metal wool and a powdered metal. The contaminated water, air or mixtures thereof are subjected to reductive and oxidative chemical species that are formed when the UV light produced by UV light source 78 is exposed to photocatalytic coated metal substrate 76. In an embodiment of the invention, sealed interior portion 72 may also include at least one mirror to reflect unabsorbed photons that are produced when the UV light is exposed to photocatalytic coated metal substrate 76. The reductive and oxidative chemical species destroy substantially all of the toxins and pathogens found in the contaminated water, air or mixtures thereof introduced into sealed interior portion 72 of container 71. After exposure to the reductive and oxidative chemical species, the decontaminated water, air or mixtures thereof flow out of container 71 through outlet 79. Although not shown, a power source provides energy to UV light sources 58, 68 and 78. In one embodiment, the power source is an electrical outlet. In yet another embodiment the power source is a battery which may be rechargeable. Although the embodiment as shown in FIG. 8 provides three containers for treating contaminated water, air or mixtures thereof, it is envisioned that more or less than three interconnected containers can be used to decontaminate water, air or mixtures thereof.

It is envisioned that the devices such as shown in FIGS. 7 and 8 and described herein, may be suitable for use in decontaminating fluids such as water and air in applications, such as including hospital or other health care environments, commercial or residential environments or air or water supplies, swimming pools, spas, purification of the air in airplanes or the like, municipal water treatment facilities or a wide variety of other applications or environments. In a further example, the coating produced on the metal substrate material may be removed from the substrate to form a photocatalytic powder type of material for use in other applications.

The invention provides a photocatalyst formed of titanium (IV) oxide, which when illuminated with UV (or visible) light produces radicals, oxidants and reductants that can function as powerful biocides. Such photocatalysts can therefore be used in decontamination of air and water by destroying toxins. The photocatalyst may be formed and retained on a substrate as described, or formed and removed from the substrate for use. The photoactive coating produced according to the invention is self-sterilizing and self-decontaminating. It is also contemplated that the coating can be altered to demonstrate an absorbance in the visible region, thus creating a visible light photocatalyst that may be used to generate hydroxyl radicals and function as a biocide or for other applications as described above, using light in the visible spectrum. The photocatalytic materials formed according to the process of the invention may be used in biocidal systems for virus inactivation using the surface coating photocatalytic system. The formed photocatalysts may also be used for synthesizing molecules, where the reductive electrons and oxidative positive holes may be used for photocatalytic conversion of molecules and can be used to synthesize molecular moieties. For example, the titanium oxide photocatalysts may be used in the production of methanol from carbon dioxide using sunlight. The photocatalysts may be used for activating and fixing carbon dioxide. Such an approach can contribute towards transforming excess carbon dioxide in the environment into useful products such as methanol. It can also oxidize oxygen or organic materials directly. Titanium dioxide formed according to the invention may thus added to paints, cements, windows, tiles, or other products for sterilizing, deodorizing and anti-fouling properties, or incorporated into outdoor building materials, such as paving stones or other products. In such applications, the products can substantially reduce concentrations of airborne pollutants such as volatile organic compounds and nitrogen oxides for example. The material may also be used as a hydrolysis catalyst, and thus be used in energy production by breaking water into hydrogen and oxygen, where the hydrogen could be collected and used as a fuel. It may also be used in types of chemical solar cells, or to produce electricity when in nanoparticle form. Nanoparticles may be useful to form the pixels of a display screen, as they generate electricity when transparent and under the influence of light, but if subjected to electricity, the nanoparticles blacken, forming the basic characteristics of a display system. The materials may also be useful in self-cleaning or anti-fogging applications as it may provide superhydrophilic characteristics when exposed to sun light. In another example, the photocatalytic coatings and/or powders could also be used to create sensors to detect certain analytes. If there is an interaction between the analyte and the surface, changes in conductance or resistance can be used to detect the presence of the analyte. Further, the oxidative, reductive and radical species generated by photocatalysts according to the invention can be used to decontaminate air and water.

Based upon the foregoing disclosure, it should now be apparent that the methods of producing photocatalytic coatings or materials provide a highly effective photocatalytic material for various possible uses. Example apparatus for the decontamination of fluids are described, but a wide variety of other devices are envisioned that may utilize the materials. It is, therefore, to be understood that any variations evident fall within the scope of the claimed invention and thus, the selection of specific component elements can be determined without departing from the spirit of the invention herein disclosed and described.

1. A process for preparing a photocatalytic material, the process comprising the steps of:
providing a metal substrate;
subjecting the metal substrate to an acid mixture;
heating the metal substrate and acid mixture to a predetermined temperature for a predetermined amount of time to form an acid treated metal substrate;
neutralizing the acid-treated metal substrate with a basic solution; and
annealing the neutralized metal substrate at a predetermined temperature for a predetermined amount of time to form a coating on the metal substrate formed of a photocatalytic material.

2. The process of claim 1, further comprising the step of cleaning the metal substrate with a cleaning solution prior to introducing the substrate into the acid mixture.

3. The process of claim 1, wherein the step of subjecting the metal substrate to an acid mixture includes positioning the metal substrate into a container and providing the acid mixture in the container.

4. (canceled)

5. The process of claim 3, wherein the container is made of borosilicate glass.

6. The process of claim 1, further comprising providing a predetermined amount of silicon oxide in the acid mixture.

7. The process of claim 1, wherein the step of heating includes exposing the metal substrate and acid mixture to a temperature of at least about 200° C. for at least about 30 minutes.

8. The process of claim 1, wherein the step of annealing the neutralized metal substrate includes exposing the neutralized metal substrate to a temperature of at least about 600° C. for at least about 60 minutes.

9. (canceled)

10. The process of claim 1, wherein the metal substrate includes titanium metal.

11. (canceled)

12. (canceled)

13. The process of claim 1, wherein the acid mixture is a combination of hydrochloric acid and nitric acid.

14. The process of claim 13, wherein the acid mixture further includes an amount of sodium fluoride.

15. The process of claim 13, wherein the mixture of hydrochloric acid and nitric acid is in the range of about 25%-75% hydrochloric acid: 75%-25% nitric acid.

16. The process of claim 15, wherein the mixture of hydrochloric acid and nitric acid is in the range of about 40%-60% hydrochloric acid: 60%-40% nitric acid.

17. The process of claim 15, wherein the mixture of hydrochloric acid and nitric acid is about 45% hydrochloric acid: 55% nitric acid.

18. The process of claim 1, further comprising the step of removing the coating from the metal substrate to provide a powder material.

19. A photocatalytic material formed according to the process of claim 1.

20. A photocatalytic substrate comprising:
a metal substrate having a surface area; and
a photocatalytic coating covering at least a portion of the surface area of the metal substrate, wherein the photocatalytic coating comprises a metal oxide coating, wherein the metal oxide coating is formed from metal ions in the metal substrate.

21. (canceled)

22. The substrate of claim 20, wherein the metal substrate includes titanium metal.

23. (canceled)

24. (canceled)

25. A device for decontaminating a fluid comprising:
at least one container comprising a sealed interior portion, an inlet providing access to the sealed interior portion and an outlet providing an exit from the sealed interior portion, wherein the sealed interior portion includes a ultra-violet source of light and a metal substrate coated with a photocatalytic coating.

26. The device of claim 25 further comprising a plurality of interconnected containers, wherein each container comprises a sealed interior portion, an inlet providing access to the sealed interior portion and an outlet providing an exit from the sealed interior portion, wherein the sealed interior portion includes a ultra-violet source of light and a photocatalytic coated substrate.

27. The device of claim 25, wherein the fluid is selected from the group consisting of air and water.

28. (canceled)

29. (canceled)

30. The coated substrate of claim 28, wherein the metal substrate includes titanium metal.

31. (canceled)

32. (canceled)
